(19) United States
(12) Patent Application Publication
(10) Pub. No.: US 2026/0174556 A1
(43) Pub. Date: Jun. 25, 2026

(54) DELIVERY DEVICE FOR A REPLACEMENT HEART VALVE IMPLANT

(71) Applicant: BOSTON SCIENTIFIC SCIMED, INC., MAPLE GROVE, MN (US)

(72) Inventors: Levi Joel Wolterstorff, Saint Paul, MN (US); James M. Anderson, Corcoran, MN (US)

(73) Assignee: BOSTON SCIENTIFIC SCIMED, INC., MAPLE GROVE, MN (US)

(21) Appl. No.: 19/430,907

(22) Filed: Dec. 23, 2025

Related U.S. Application Data
(60) Provisional application No. 63/738,100, filed on Dec. 23, 2024.

(51) Int. Cl. A61F 2/24; A61F 2/95; A61F 2/962; A61M 25/06

(52) U.S. Cl. CPC A61F 2/2436; A61F 2/2418; A61F 2/9517; A61F 2/962; A61F 2210/0014; A61F 2230/0069; A61M 2025/0681

(57) ABSTRACT

A delivery device for a replacement heart valve implant includes a handle assembly and a shaft assembly extending from the handle. The shaft assembly includes an inner shaft, an outer tubular member, and an implant holding portion including proximal and distal sheaths. In a delivery configuration, the holding portion constrains the implant within the proximal and distal sheaths. In a withdrawal configuration, the proximal sheath axially overlaps the distal sheath. The holding portion includes a stent holder having a proximal head, a distal tail, and a body portion. In the delivery configuration, the distal sheath constrains the implant in engagement with the proximal head. A method of delivering the implant includes advancing the holding portion to a native heart valve, translating the proximal and distal sheaths to release proximal and distal portions of the implant, and translating the proximal sheath until the proximal and distal sheaths axially overlap.

### CROSS-REFERENCE TO RELATED APPLICATIONS

This application claims the benefit of priority of U.S. Provisional Application No. 63/738,100 filed Dec. 23, 2024, the entire disclosure of which is hereby incorporated by reference.

### TECHNICAL FIELD

The disclosure relates generally to medical devices and more particularly to medical devices that are adapted for implanting stents and medical devices including a stent component, such as a replacement heart valve implant.

### BACKGROUND

A wide variety of intracorporeal medical devices and/or implants have been developed for medical use including artificial heart valve implants for repair or replacement of diseased heart valves. Some delivery devices may require a number of steps in a particular order to properly deploy an implant. Some users may perform a low volume of cases, and familiarity with the delivery device may be limited. Confusion, cognitive load, and/or stress may slow and/or complicate the procedure. Of the known medical devices, systems, and methods, each has certain advantages and disadvantages. There is an ongoing need to provide alternative devices, systems, and methods for loading medical devices and/or heart valve implants into a delivery system.

### SUMMARY

In one example, a delivery device for delivering a replacement heart valve implant to a native heart valve may comprise a handle assembly, and an elongate shaft assembly extending distally from the handle assembly. The elongate shaft assembly may comprise an inner shaft secured to the handle assembly, an outer tubular member disposed around the inner shaft and secured to the handle assembly, and an implant holding portion comprising a proximal sheath and a distal sheath. In a delivery configuration of the implant holding portion, the implant holding portion may be configured to constrain the replacement heart valve implant within the proximal sheath and the distal sheath. In a withdrawal configuration of the implant holding portion, at least a portion of the proximal sheath axially may overlap at least a portion of the distal sheath.

In addition, or alternatively, to any example disclosed herein, the proximal sheath is axially offset from the distal sheath in the delivery configuration.

In addition, or alternatively, to any example disclosed herein, the proximal sheath is axially non-overlapping with the distal sheath in the delivery configuration.

In addition, or alternatively, to any example disclosed herein, the distal sheath comprises a tubular portion having a constant inner diameter and a tapered portion disposed at a proximal end of the tubular portion, wherein an inner surface of the tapered portion tapers radially inward in a proximal direction.

In addition, or alternatively, to any example disclosed herein, an outer surface of the tapered portion tapers radially inward in the proximal direction such that at a proximalmost end of the tapered portion an outer diameter of the tapered portion is less than an inner diameter of the proximal sheath at a distalmost end of the proximal sheath.

In addition, or alternatively, to any example disclosed herein, the tapered portion comprises a plurality of longitudinally oriented spines formed from a different material than the tapered portion.

In addition, or alternatively, to any example disclosed herein, the implant holding portion is configured to shift from the delivery configuration to a deployment configuration wherein the proximal sheath and the distal sheath are axially spaced apart by a distance greater than an overall length of the replacement heart valve implant to release the replacement heart valve implant.

In addition, or alternatively, to any example disclosed herein, and in a second example, a delivery device for delivering a replacement heart valve implant to a native heart valve may comprise a handle assembly, and an elongate shaft assembly extending distally from the handle assembly. The elongate shaft assembly may comprise an inner shaft secured to the handle assembly, an outer tubular member disposed around the inner shaft and secured to the handle assembly, and an implant holding portion comprising a stent holder, a proximal sheath, and a distal sheath. The stent holder may comprise a proximal head having a first outermost radial extent, a distal tail having a second outermost radial extent, and a body portion extending from the proximal head to the distal tail, the body portion having a constant outer diameter less than the first outermost radial extent and the second outermost radial extent. In a delivery configuration of the implant holding portion, the distal sheath may be configured to constrain at least a portion of the replacement heart valve implant in engagement with the proximal head of the stent holder.

In addition, or alternatively, to any example disclosed herein, the distal sheath comprises a tubular portion having a constant inner diameter and a tapered portion disposed at a proximal end of the tubular portion, wherein the tapered portion is self-biased radially inward.

In addition, or alternatively, to any example disclosed herein, in the delivery configuration of the implant holding portion, the tapered portion is biased radially outward by the replacement heart valve implant.

In addition, or alternatively, to any example disclosed herein, in a deployment configuration of the implant holding portion, the tapered portion is disposed distal of the replacement heart valve implant and is in contact with the body portion of the stent holder.

In addition, or alternatively, to any example disclosed herein, in a withdrawal configuration of the implant holding portion, the proximal sheath is in contact with a radially outward facing surface of the tapered portion.

In addition, or alternatively, to any example disclosed herein, the tapered portion is formed from an elastomeric material.

In addition, or alternatively, to any example disclosed herein, the tubular portion comprises a reinforcing element configured to prevent radial expansion of the tubular portion.

In addition, or alternatively, to any example disclosed herein, in the delivery configuration of the implant holding portion, the proximal head of the stent holder is disposed within the distal sheath, and in a withdrawal configuration of the implant holding portion, the proximal head of the stent holder is disposed within the proximal sheath.

In addition, or alternatively, to any example disclosed herein, and in a third example, a method of delivering a replacement heart valve implant to a native heart valve of a patient may comprise advancing an implant holding portion of a delivery device, wherein the implant holding portion comprises a proximal sheath and a distal sheath constraining the replacement heart valve implant therein, to the native heart valve; translating the proximal sheath in a proximal direction relative to the replacement heart valve implant to release a proximal portion of the replacement heart valve implant; translating the distal sheath in a distal direction relative to the replacement heart valve implant to release a distal portion of the replacement heart valve implant; translating the proximal sheath in the distal direction until at least a portion of the proximal sheath axially overlaps at least a portion of the distal sheath; and removing the delivery device from the patient.

In addition, or alternatively, to any example disclosed herein, the method may comprise: prior to translating the proximal sheath in the distal direction, retracting the delivery device relative to the replacement heart valve implant until a proximal end of the distal sheath is disposed proximal of valve leaflets of the replacement heart valve implant.

In addition, or alternatively, to any example disclosed herein, the implant holding portion comprises a stent holder comprising a proximal head having a first outermost radial extent, a distal tail having a second outermost radial extent, and a body portion extending from the proximal head to the distal tail, the body portion having a constant outer diameter less than the first outermost radial extent and the second outermost radial extent.

In addition, or alternatively, to any example disclosed herein, the distal sheath comprises a tubular portion and a tapered portion disposed at a proximal end of the distal sheath, and the tapered portion extends proximal of the body portion of the stent holder prior to releasing the replacement heart valve implant.

In addition, or alternatively, to any example disclosed herein, translating the distal sheath in the distal direction positions a proximalmost end of the tapered portion in contact with the body portion of the stent holder.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a replacement heart valve implant;

FIGS. 2-3 schematically illustrate selected aspects of a replacement heart valve system including the replacement heart valve implant and a delivery device;

FIG. 4 is a partial cross-sectional view illustrating selected aspects of the delivery device;

FIGS. 5-6 schematically illustrate selected aspects of a distal sheath of the delivery device;

FIGS. 7A-7C are partial cutaway views illustrating selected aspects of a tapered portion of the distal sheath of the delivery device of FIGS. 5-6; and

FIGS. 8-12 schematically illustrate selected aspects related to a method of delivering the replacement heart valve implant to a native heart valve.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

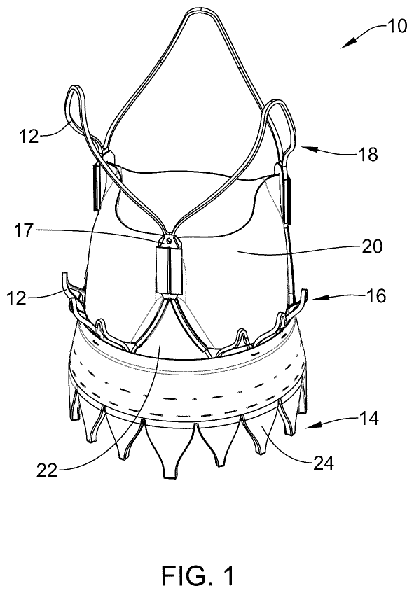

FIG. 1 illustrates selected aspects of a replacement heart valve implant 10. It should be appreciated that the replacement heart valve implant 10 can be any type of replacement heart valve (e.g., a mitral valve, an aortic valve, etc.). Some non-limiting examples of the replacement heart valve implant 10 may include the ACURATE NEO2™, the ACURATE PRIME™, and/or family members thereof from Boston Scientific. Other examples are also contemplated. In use, the replacement heart valve implant 10 may be implanted (e.g., surgically or through transcatheter delivery) within a native heart valve in a mammalian heart. The replacement heart valve implant 10 can be configured to allow one-way flow through the replacement heart valve implant 10 from an inflow end to an outflow end.

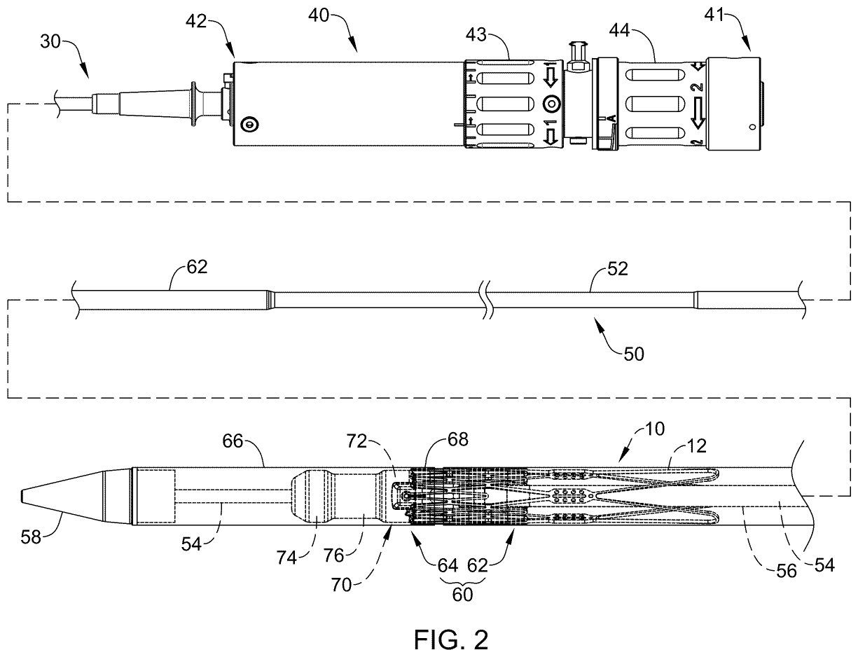

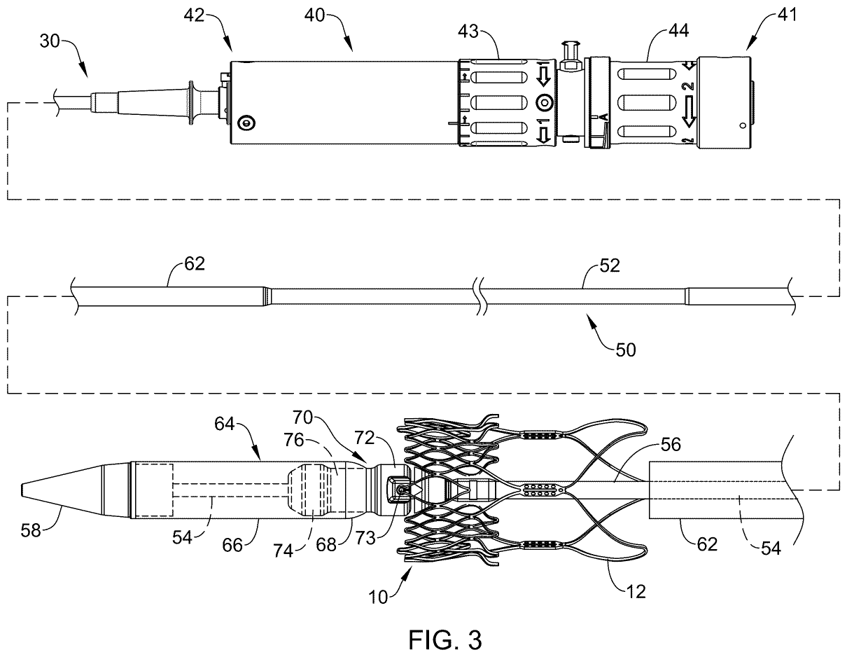

The replacement heart valve implant 10 may include an expandable framework 12 defining a central lumen. In some embodiments, the expandable framework 12 may have a substantially circular cross-section. In some embodiments, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The replacement heart valve implant 10 and/or the expandable framework 12 may be configured to shift between a radially collapsed configuration (e.g., FIG. 2) and a radially expanded configuration (e.g., FIG. 3). In some embodiments, the expandable framework 12 may be self-expanding. In some embodiments, the expandable framework 12 may be self-biased toward the radially expanded configuration. In some embodiments, the expandable framework 12 may be mechanically expandable. In some embodiments, the expandable framework 12 may be balloon expandable. Other configurations, including combinations thereof, are also contemplated.

In some embodiments, the expandable framework 12 may define a lower crown 14 proximate and/or at an inflow end, an upper crown 16 proximate and/or at an outflow end, and a plurality of stabilization arches 18 extending downstream from the outflow end. The expandable framework may comprise a body portion extending between the lower crown 14 and the upper crown 16. In at least some embodiments, the body portion may be formed as and/or similar to a stent or a stent-like structure. In some embodiments, the body portion may comprise a plurality of closed cells. In some embodiments, the expandable framework 12 may comprise a plurality of commissure posts 17. In some embodiments, the plurality of commissure posts 17 may extend downstream of and/or away from the upper crown 16. In some embodiments, the plurality of stabilization arches 18 may extend downstream of and/or away from the upper crown 16 and/or the plurality of commissure posts 17 in a direction opposite the lower crown 14. In some embodiments, the upper crown 16 and/or the plurality of commissure posts 17 may be disposed longitudinally and/or axially between the lower crown 14 and the plurality of stabilization arches 18.

In some embodiments, the replacement heart valve implant 10 and/or the expandable framework 12 may include a proximal portion and a distal portion. In some embodiments, orientation of the replacement heart valve implant 10 may be related to a delivery device 30 (e.g., FIGS. 2-3) and/or a direction of implantation relative to a treatment site (e.g., a native heart valve). In some embodiments, the proximal portion may include the outflow end and/or the plurality of stabilization arches 18. In some embodiments, the proximal portion may include the upper crown 16 and/or the plurality of commissure posts 17. In some embodiments, the distal portion may include the inflow end and/or the lower crown 14. In some embodiments, the distal portion may include the body portion and/or a portion of the body portion disposed immediately adjacent the lower crown 14. Other configurations are also contemplated.

In some embodiments, the replacement heart valve implant 10 may include a plurality of valve leaflets 20 disposed within the central lumen. The plurality of valve leaflets 20 may be coupled, secured, and/or fixedly attached to the expandable framework 12 at the plurality of commissure posts 17 to form and/or define a plurality of commissures. In addition, or alternatively, in some embodiments, the plurality of valve leaflets 20 may be coupled, secured, and/or fixedly attached to the expandable framework 12 proximate and/or at other locations, such as the inflow end, the lower crown 14, etc. The plurality of valve leaflets 20 may be configured to shift between an open position and a closed position. The plurality of valve leaflets 20 may be configured to substantially restrict fluid flow through the replacement heart valve implant 10 in the closed position. The plurality of valve leaflets 20 may move apart from each other and/or radially outward within the central lumen in the open position to permit fluid flow through the replacement heart valve implant 10 and/or the central lumen.

In some embodiments, the plurality of valve leaflets 20 may be comprised of a polymer, such as a thermoplastic polymer. In some embodiments, the plurality of valve leaflets 20 may include at least 50 percent by weight of a polymer. In some embodiments, the plurality of valve leaflets 20 may be formed from porcine pericardium, bovine pericardium, or other tissue. Other configurations and/or materials are also contemplated.

In some embodiments, the replacement heart valve implant 10 may include an inner skirt 22 disposed on and/or extending along an inner surface of the expandable framework 12. In at least some embodiments, the inner skirt 22 may be fixedly attached to the expandable framework 12. The inner skirt 22 may direct fluid, such as blood, flowing through the replacement heart valve implant 10 toward the plurality of valve leaflets 20. In at least some embodiments, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some embodiments, the replacement heart valve implant 10 may include an outer skirt 24 disposed on and/or extending along an outer surface of the expandable framework 12. In some embodiments, the outer skirt 24 may be disposed at and/or adjacent the lower crown 14. The outer skirt 24 may ensure the fluid flows through the replacement heart valve implant 10 and does not flow around the replacement heart valve implant 10 (e.g., between the expandable framework 12 and the vessel wall).

In some embodiments, the inner skirt 22 and/or the outer skirt 24 may include a polymer, and/or may include at least 50 percent by weight of a polymer. In some embodiments, the inner skirt 22 and/or the outer skirt 24 may be substantially impervious to fluid. In some embodiments, the inner skirt 22 and/or the outer skirt 24 may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

In some embodiments, the inner skirt 22 and/or the outer skirt 24 may seal one of, some of, a plurality of, or each of a plurality of interstices formed in the expandable framework 12. In at least some embodiments, sealing the interstices may be considered to prevent fluid from flowing through the interstices of the expandable framework 12. In some embodiments, the inner skirt 22 and/or the outer skirt 24 may be attached to the expandable framework 12 using one or more methods including but not limited to tying with sutures or filaments, adhesive bonding, melt bonding, embedding or over molding, welding, etc.

In some embodiments, the expandable framework 12 and/or the replacement heart valve implant 10 may have an outer extent of about 17 mm (mm) (0.669 inches), about 19.5 mm (0.768 inches), about 23 millimeters (0.905 inches), about 25 mm (0.984 inches), about 27 mm (1.063 inches), about 30 mm (1.181 inches), about 31 mm (1.220 inches), etc. in an unconstrained configuration (e.g., in the radially expanded configuration). In some embodiments, the expandable framework 12 and/or the replacement heart valve implant 10 may have an outer extent of about 11 mm (0.433 inches), about 10 mm (0.394 inches), about 9 mm (0.354 inches), about 8 mm (0.315 inches), about 7 mm (0.276 inches), about 6 mm (0.236 inches), about 5 mm (0.197 inches), about 4 mm (0.157 inches), etc. in the radially collapsed configuration. Other configurations are also contemplated.

FIGS. 2-4 illustrate selected aspects of a replacement heart valve system comprising the replacement heart valve implant 10 and a delivery device 30 for delivering the replacement heart valve implant 10 to a treatment site (e.g., native heart valve). It should be noted that FIGS. 2-4 may include at least one change of scale (e.g., all parts of the figure are not drawn to the same scale) to improve viewability and show additional detail of selected aspects of the delivery device 30. Additionally, the expandable framework 12 is shown and some elements of the replacement heart valve implant 10 are omitted from FIGS. 2-3 to improve clarity. It should further be noted that all elements are not shown or labelled in each figure in the interest of clarity.

The delivery device 30 may include a handle assembly 40 and an elongate shaft assembly 50 extending distally from the handle assembly 40. The handle assembly 40 may include a proximal end 41 and a distal end 42 opposite the proximal end 41. The elongate shaft assembly 50 may extend distally from the distal end 42 of the handle assembly 40. In some embodiments, the handle assembly 40 may include one or more rotatable knobs. In some embodiments, the one or more rotatable knobs may include a first rotatable knob 43 and a second rotatable knob 44. In at least some embodiments, the first rotatable knob 43 and/or the second rotatable knob 44 may be configured to rotate around a central longitudinal axis of the delivery device 30 and/or the handle assembly 40. Other configurations and/or types of interface controls are also contemplated.

In some embodiments, rotation of the first rotatable knob 43 around the central longitudinal axis of the handle assembly 40 may be configured to deploy the proximal portion of the replacement heart valve implant 10 from an implant holding portion 60 of the delivery device 30. Similarly, rotation of the second rotatable knob 44 around the central longitudinal axis of the handle assembly 40 may be configured to deploy the distal portion of the replacement heart valve implant 10 from the implant holding portion 60 of the delivery device 30.

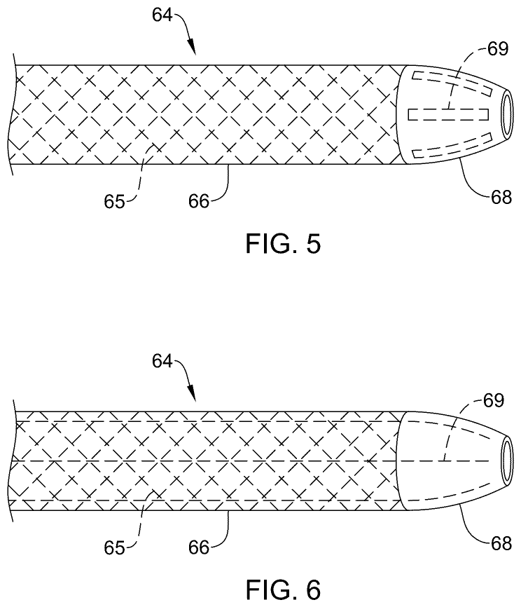

In some embodiments, a distal portion of the delivery device 30 and/or the elongate shaft assembly 50 may comprise the implant holding portion 60. The implant holding portion 60 may be configured to engage with and/or constrain the replacement heart valve implant 10 and/or the expandable framework 12 in the radially collapsed configuration. The elongate shaft assembly 50 may include an outer tubular member 52 secured to and extending distally from the handle assembly 40, and an inner shaft 54 secured to and extending distally from the handle assembly 40 within the outer tubular member 52 to a distal tip 58 disposed distal of the implant holding portion 60. In some embodiments, the implant holding portion 60 may comprise a proximal sheath 62 and a distal sheath 64. In some embodiments, in a delivery configuration of the implant holding portion 60 (e.g., FIG. 2), the implant holding portion 60 may be configured to constrain the replacement heart valve implant 10 and/or the expandable framework 12 within the proximal sheath 62 and the distal sheath 64. In some embodiments, the proximal sheath 62 and/or the distal sheath 64 may be formed from a polymeric material. In some embodiments, the proximal sheath 62 and/or the distal sheath 64 may include a reinforcing element 65 (e.g., FIGS. 5-6) disposed therein and/or thereon. In some embodiments, the reinforcing element 65 (e.g., FIGS. 5-6) may be a coil, a mesh, one or more filaments, bands, or strips, or another suitable structure. The reinforcing element 65 is not shown in FIGS. 2-4 to improve clarity, but it shall be understood that the reinforcing element 65 may be present. Other configurations are also contemplated. In some embodiments, the reinforcing element 65 (e.g., FIGS. 5-6) may be configured to prevent radial expansion of the distal sheath 64.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and about, around, surrounding, and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 may comprise a tubular portion 66 and a tapered portion 68 disposed at a proximal end of the tubular portion 66. In at least some embodiments, the tubular portion 66 may have a constant inner diameter. In some embodiments, the tubular portion 66 may comprise the reinforcing element 65 (e.g., FIGS. 5-6) discussed herein disposed in or on the tubular portion 66 of the distal sheath 64. As such, the reinforcing element 65 (e.g., FIGS. 5-6) may be configured to prevent radial expansion of the tubular portion 66 of the distal sheath 64. In some embodiments, the tapered portion 68 may be devoid of the reinforcing element 65 (e.g., FIGS. 5-6) disposed in or on the tubular portion 66 of the distal sheath 64.

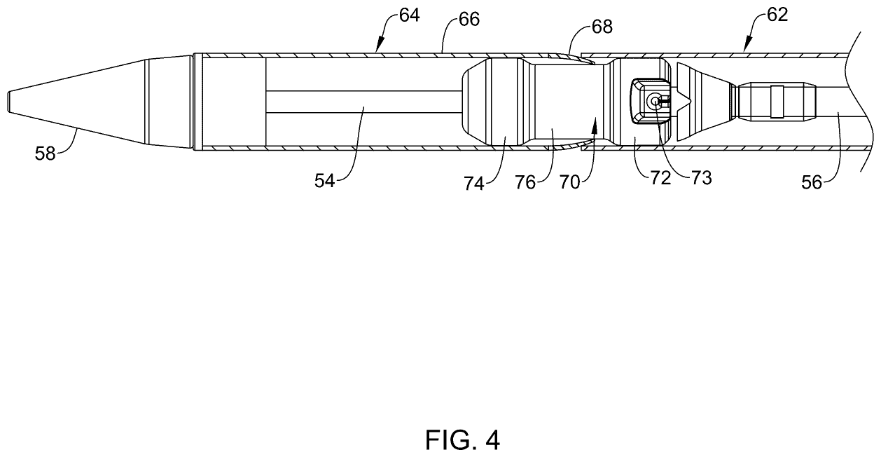

In some embodiments, the tapered portion 68 of the distal sheath 64 may be fixedly attached to the tubular portion 66 of the distal sheath 64. In some embodiments, the tapered portion 68 of the distal sheath 64 may be formed from an elastic material and/or an elastomeric material. In some embodiments, the tapered portion 68 of the distal sheath 64 may be fixedly attached to the tubular portion 66 of the distal sheath 64 via reflow, welding, adhesive bonding, etc. In some embodiments, an outer surface of the tapered portion may taper radially inward in a proximal direction, as seen in FIGS. 3-4, such that at a proximalmost end of the tapered portion 68 an outer diameter of the tapered portion is less than an outer diameter of the proximal sheath 62 at a distalmost end of the proximal sheath 62 and/or the outer diameter of the tapered portion is less than an outer diameter of the tubular portion 66 of the distal sheath 64 at the proximal end of the tubular portion 66. In some embodiments, an inner surface of the tapered portion 68 may taper radially inward in a proximal direction, as seen in FIG. 4. In some embodiments, the tapered portion 68 of the distal sheath 64 may be self-biased radially inward.

In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle assembly 40.

In some embodiments, the handle assembly 40 may be configured to manipulate and/or translate the proximal sheath 62 and/or the distal sheath 64 relative to each other using the first rotatable knob 43 and/or the second rotatable knob 44, as discussed further below, to shift the implant holding portion 60, and/or the proximal sheath 62 and the distal sheath 64, between a delivery configuration (e.g., FIG. 2), a deployment configuration (e.g., FIG. 3), and a withdrawal configuration (e.g., FIG. 4). In some embodiments, the handle assembly 40 and/or the second rotatable knob 44 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle assembly 40 and/or the first rotatable knob 43 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 10 to a treatment site (e.g., the native heart valve, the aortic valve, etc.), the replacement heart valve implant 10 may be disposed at least partially within the proximal sheath 62 and/or the distal sheath 64 in the radially collapsed configuration in the delivery configuration of the implant holding portion 60. In some embodiments, the proximal sheath 62 and/or the distal sheath 64 may collectively define the implant holding portion 60 of the delivery device 30. In some embodiments, the implant holding portion 60 may be configured to constrain the replacement heart valve implant 10 in the radially collapsed configuration when the implant holding portion 60 is in the delivery configuration.

In some embodiments, the implant holding portion 60 of the delivery device 30 may comprise a stent holder 70. In some embodiments, the replacement heart valve implant 10 may be releasably coupled to and/or releasably engaged with the intermediate tubular member 56 and/or the stent holder 70 when the replacement heart valve implant 10 is constrained within the implant holding portion 60 of the delivery device 30 in the radially collapsed configuration.

In some embodiments, the stent holder 70 may comprise a proximal head 72 having a first outermost radial extent, a distal tail 74 having a second outermost radial extent, and a body portion 76 extending from the proximal head 72 to the distal tail 74. The body portion 76 may have an outer diameter less than the first outermost radial extent and the second outermost radial extent. In at least some embodiments, the body portion 76 may have a constant outer diameter less than the first outermost radial extent and the second outermost radial extent along its entire length. Other configurations are also contemplated.

In some embodiments, the stent holder 70 may be fixedly attached to the elongate shaft assembly 50. In some embodiments, the stent holder 70 may be fixedly attached to the intermediate tubular member 56 of the elongate shaft assembly 50. In some alternative embodiments, the stent holder 70 may be monolithically formed with the elongate shaft assembly 50 and/or the intermediate tubular member 56. In some embodiments, the stent holder 70 may be configured to engage the expandable framework 12 in the radially collapsed configuration and/or when the replacement heart valve implant 10 is constrained within the implant holding portion 60 of the delivery device 30. In some embodiments, the stent holder 70 and/or the proximal head 72 may include at least one projection 73 configured to engage the expandable framework 12 and/or the lower crown 14 in the radially collapsed configuration.

In some embodiments, in the delivery configuration of the implant holding portion 60, the distal sheath 64 may be configured to constrain at least a portion of the replacement heart valve implant 10 (e.g., at least a portion of the expandable framework 12 and/or the lower crown 14) in engagement with the proximal head 72 and/or the at least one projection 73 of the stent holder 70. In some embodiments, in the delivery configuration of the implant holding portion 60, the tubular portion 66 and/or the tapered portion 68 of the distal sheath 64 may be configured to constrain at least a portion of the replacement heart valve implant 10 (e.g., at least a portion of the expandable framework 12 and/or the lower crown 14) in engagement with the proximal head 72 and/or the at least one projection 73 of the stent holder 70. In some embodiments, in the delivery configuration of the implant holding portion 60, the proximal head 72 of the stent holder 70 may be disposed within the distal sheath 64. In some embodiments, in the delivery configuration of the implant holding portion 60, the proximal head 72 of the stent holder 70 may be disposed within the tapered portion 68 and/or the tubular portion 66 of the distal sheath 64.

In some embodiments, in the delivery configuration of the implant holding portion 60, the tapered portion 68 of the distal sheath 64 may be biased radially outward by the replacement heart valve implant 10, as seen in FIG. 2. For example, the tapered portion 68 may be elastically stretched and/or deformed radially outward by the replacement heart valve implant 10 disposed therein.

In some embodiments, the proximal sheath 62 may be configured to cover the proximal portion of the replacement heart valve implant 10 in the radially collapsed configuration when the implant holding portion 60 is in the delivery configuration, and the distal sheath 64 may be configured to cover the distal portion of the replacement heart valve implant 10 in the radially collapsed configuration when the implant holding portion 60 is in the delivery configuration. In some embodiments, the replacement heart valve implant 10 may be constrained in the radially collapsed configuration by the proximal sheath 62 and the distal sheath 64 in the delivery configuration of the implant holding portion 60. In some embodiments, the proximal sheath 62 may be disposed adjacent to the distal sheath 64 in the delivery configuration. In some embodiments, the proximal sheath 62 may abut the distal sheath 64 in the delivery configuration. In some embodiments, the proximal sheath 62 may be axially offset from the distal sheath 64 in the delivery configuration. In some embodiments, the proximal sheath 62 may be axially non-overlapping with the distal sheath 64 in the delivery configuration. In some embodiments, the proximal sheath 62 may be axially spaced apart from the distal sheath 64 in the delivery configuration.

In some embodiments, the proximal sheath 62 may be axially spaced apart from the distal sheath 64 in the delivery configuration by less than 20% of an overall length of the replacement heart valve implant 10 in the radially collapsed configuration. In some embodiments, the proximal sheath 62 may be axially spaced apart from the distal sheath 64 in the delivery configuration by less than 15% of an overall length of the replacement heart valve implant 10 in the radially collapsed configuration. In some embodiments, the proximal sheath 62 may be axially spaced apart from the distal sheath 64 in the delivery configuration by less than 10% of an overall length of the replacement heart valve implant 10 in the radially collapsed configuration. In some embodiments, the proximal sheath 62 may be axially spaced apart from the distal sheath 64 in the delivery configuration by less than 5% of an overall length of the replacement heart valve implant 10 in the radially collapsed configuration. Other configurations are also contemplated.

In some embodiments, the handle assembly 40 comprises an actuation mechanism operatively engaged with the first rotatable knob 43. In some embodiments, the first rotatable knob 43 may be fixedly attached to the actuation mechanism. In some embodiments, the first rotatable knob 43 may be configured to rotate the actuation mechanism relative to the central longitudinal axis of the handle assembly 40. In some embodiments, rotation of the first rotatable knob 43 around the central longitudinal axis of the handle assembly 40 causes rotation of and/or rotates the actuation mechanism around the central longitudinal axis of the handle assembly 40.

In some embodiments, the first rotatable knob 43 may be configured to rotate the actuation mechanism relative to the central longitudinal axis of the handle assembly 40 to axially translate a first portion of the elongate shaft assembly 50 along the central longitudinal axis. In some embodiments, the first portion of the elongate shaft assembly 50 may comprise and/or may be the outer tubular member 52 and/or the proximal sheath 62. In some embodiments, rotation of the first rotatable knob 43 and/or the actuation mechanism may be configured to axially translate the first portion (e.g., the outer tubular member 52 and/or the proximal sheath 62) of the elongate shaft assembly 50 relative to the handle assembly 40. In some embodiments, rotation of the first rotatable knob 43 and/or the actuation mechanism in a first rotational direction may be configured to axially translate the first portion (e.g., the outer tubular member 52 and/or the proximal sheath 62) of the elongate shaft assembly 50 distally relative to the handle assembly 40 and/or toward the delivery configuration of the implant holding portion 60 (e.g., FIG. 2) and/or the withdrawal configuration of the implant holding portion 60 (e.g., FIG. 4). In some embodiments, rotation of the first rotatable knob 43 and/or the actuation mechanism in a second rotational direction (e.g., opposite the first rotational direction) may be configured to axially translate the first portion (e.g., the outer tubular member 52 and/or the proximal sheath 62) of the elongate shaft assembly 50 proximally relative to the handle assembly 40 and/or toward the deployment configuration of the implant holding portion 60 (e.g., FIG. 3). In at least some embodiments, the first rotational direction may be clockwise as viewed from the proximal end 41 of the handle assembly 40 to the distal end 42 of the handle assembly 40, and the second rotational direction may be counterclockwise as viewed from the proximal end 41 of the handle assembly 40 toward the distal end 42 of the handle assembly 40.

In some embodiments, the handle assembly 40 may comprise a second actuation mechanism operatively engaged with the second rotatable knob 44. In some embodiments, the second rotatable knob 44 may be fixedly attached to the second actuation mechanism. In some embodiments, the second rotatable knob 44 may be configured to rotate the second actuation mechanism relative to the central longitudinal axis of the handle assembly 40. In some embodiments, rotation of the second rotatable knob 44 around the central longitudinal axis of the handle assembly 40 causes rotation of and/or rotates the second actuation mechanism around the central longitudinal axis of the handle assembly 40.

In some embodiments, the second rotatable knob 44 may be configured to rotate the second actuation mechanism relative to the central longitudinal axis of the handle assembly 40 to axially translate a second portion of the elongate shaft assembly 50 along the central longitudinal axis. In some embodiments, the second portion of the elongate shaft assembly 50 may comprise and/or may be the inner shaft 54 and/or the distal sheath 64. In some embodiments, rotation of the second rotatable knob 44 and/or the second actuation mechanism may be configured to axially translate the second portion (e.g., the inner shaft 54 and/or the distal sheath 64) of the elongate shaft assembly 50 relative to the handle assembly 40. In some embodiments, rotation of the second rotatable knob 44 and/or the second actuation mechanism in the first rotational direction may be configured to axially translate the second portion (e.g., the inner shaft 54 and/or the distal sheath 64) of the elongate shaft assembly 50 proximally relative to the handle assembly 40 and/or toward the delivery configuration of the implant holding portion 60 (e.g., FIG. 2). In some embodiments, rotation of the second rotatable knob 44 and/or the second actuation mechanism in the second rotational direction (e.g., opposite the first rotational direction) may be configured to axially translate the second portion (e.g., the inner shaft 54 and/or the distal sheath 64) of the elongate shaft assembly 50 distally relative to the handle assembly 40 and/or toward the deployment configuration of the implant holding portion 60 (e.g., FIG. 3). In some embodiments, axially translating the second portion (e.g., the inner shaft 54 and/or the distal sheath 64) of the elongate shaft assembly 50 in the distal direction to the deployment configuration may position a proximalmost end of the tapered portion 68 and/or the distal sheath 64 distal of the proximal head 72 of the stent holder 70 and/or in direct contact with the body portion 76 of the stent holder 70.

In at least some embodiments, a position of the distal sheath 64 in the deployment configuration (e.g., FIG. 3) of the implant holding portion 60 may also correspond to the position of the distal sheath 64 in the withdrawal configuration (e.g., FIG. 4) of the implant holding portion 60. Accordingly, the distal sheath 64 may remain stationary and/or does not move when shifting the implant holding portion 60 from the deployment configuration (e.g., FIG. 3) of the implant holding portion 60 toward and/or to the withdrawal configuration (e.g., FIG. 4) of the implant holding portion 60.

In some embodiments, after shifting the implant holding portion 60 to the deployment configuration (e.g., FIG. 3) and/or after releasing the replacement heart valve implant 10, the implant holding portion 60 may be shifted from the deployment configuration (e.g., FIG. 3) of the implant holding portion 60 toward and/or to the withdrawal configuration (e.g., FIG. 4) of the implant holding portion 60 to facilitate withdrawal of the delivery device 30 from the patient. In some embodiments, in the withdrawal configuration (e.g., FIG. 4) of the implant holding portion 60, at least a portion of the proximal sheath 62 may axially overlap at least a portion of the distal sheath 64, as seen in FIG. 4. In some embodiments, in the withdrawal configuration (e.g., FIG. 4) of the implant holding portion 60, at least a portion of the proximal sheath 62 may axially overlap at least a portion of the tapered portion 68 of the distal sheath 64. In some embodiments, in the withdrawal configuration (e.g., FIG. 4) of the implant holding portion 60, the distalmost end of the proximal sheath 62 may be in direct contact with the tapered portion 68 of the distal sheath 64. In some embodiments, in the withdrawal configuration (e.g., FIG. 4) of the implant holding portion 60, the distalmost end of the proximal sheath 62 may be in direct contact with the outer surface and/or a radially outward facing surface of the tapered portion 68 of the distal sheath 64. In some embodiments, in the withdrawal configuration of the implant holding portion 60, the proximal head 72 of the stent holder 70 may be disposed within the proximal sheath 62.

Turning now to FIGS. 5-6, the figures schematically illustrate that the distal sheath 64 may comprise the tubular portion 66 and the tapered portion 68. In some embodiments, the distal sheath 64 and/or the tubular portion 66 may optionally comprise the reinforcing element 65. In some embodiments, the tapered portion 68 may comprise a plurality of longitudinally oriented spines 69. In at least some embodiments, the plurality of longitudinally oriented spines 69 may be formed from a different material than the tapered portion 68. In some embodiments, the plurality of longitudinally oriented spines 69 may be formed from a metallic material. In some embodiments, the plurality of longitudinally oriented spines 69 may be formed from a polymeric material. Some suitable but non-limiting examples of materials for the plurality of longitudinally oriented spines 69, such as metallic materials, polymeric materials, composite materials, combinations thereof, and the like, are described below. In some embodiments, the plurality of longitudinally oriented spines 69 may be stiffer and/or less flexible than the tapered portion 68. In some embodiments, the plurality of longitudinally oriented spines 69 may be configured to add axial support and/or column strength to the tapered portion 68 without compromising or limiting the ability of the tapered portion 68 to elastically stretch radially outward and radially inward.

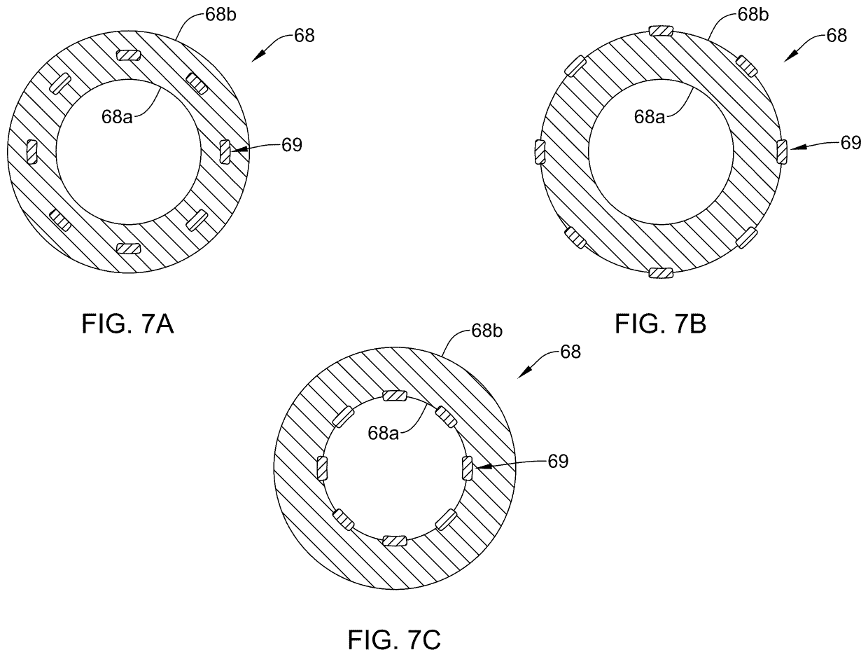

FIGS. 7A-7C are partial cutaway views illustrating selected aspects of the tapered portion 68 of the distal sheath 64 of FIGS. 5-6. In some embodiments, the plurality of longitudinally oriented spines 69 may be embedded within the tapered portion 68, as seen in the cross-sectional view of FIG. 7A. As such, the plurality of longitudinally oriented spines 69 may be disposed between an inner surface 68A and an outer surface 68B of the tapered portion 68. In some embodiments, the plurality of longitudinally oriented spines 69 may be disposed along the outer surface 68B of the tapered portion 68, as seen in the cross-sectional view of FIG. 7B. In some embodiments, the plurality of longitudinally oriented spines 69 may be disposed along the inner surface 68A of the tapered portion 68, as seen in the cross-sectional view of FIG. 7C. Other configurations are also contemplated.

In at least some embodiments, the plurality of longitudinally oriented spines 69 may be circumferentially spaced apart around the central longitudinal axis of the elongate shaft assembly 50 and/or the distal sheath 64. In some embodiments, the plurality of longitudinally oriented spines 69 may be formed as discrete segments disposed and/or contained entirely within and/or on the tapered portion 68, as seen in FIG. 5. In some embodiments, the plurality of longitudinally oriented spines 69 may extend distal of the tapered portion 68. In some embodiments, the tubular portion 66 of the distal sheath 64 may comprise a distal portion of the plurality of longitudinally oriented spines 69. In some embodiments, the plurality of longitudinally oriented spines 69 may be embedded within the tubular portion 66 of the distal sheath 64. In some embodiments, the plurality of longitudinally oriented spines 69 may be disposed along the outer surface of the tubular portion 66 of the distal sheath 64. In some embodiments, the plurality of longitudinally oriented spines 69 may be disposed along the inner surface of the tubular portion 66 of the distal sheath 64. In some embodiments, positioning of the plurality of longitudinally oriented spines 69 within and/or on the tubular portion 66 may be commensurate with (e.g., the same as) the positioning of the plurality of longitudinally oriented spines 69 within and/or on the tapered portion 68. Other configurations are also contemplated.

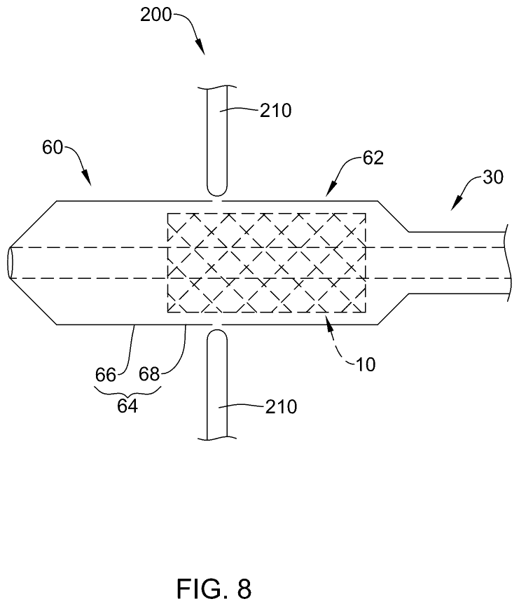

FIGS. 8-12 schematically illustrates selected aspects of a method of delivering the replacement heart valve implant 10 to a native heart valve 200 (e.g., the aortic valve) of a patient. For simplicity, the native heart valve 200 is illustrated schematically via valve leaflets 210. In some embodiments, the method of delivering the replacement heart valve implant 10 to the native heart valve 200 of the patient may comprise advancing the implant holding portion 60 of the delivery device 30 to the native heart valve 200, as seen in FIG. 8. As discussed herein, the implant holding portion 60 may comprise the proximal sheath 62 and the distal sheath 64 constraining the replacement heart valve implant 10 therein in the radially collapsed configuration. In some embodiments, the plurality of stabilization arches 18 (e.g., FIG. 1) may be positioned proximal and/or downstream of the native heart valve 200 and/or the valve leaflets 210. In some embodiments, the lower crown 14 and/or the distal portion of the replacement heart valve implant 10 and/or the expandable framework 12 may be positioned distal and/or upstream of the native heart valve 200 and/or the valve leaflets 210.

In at least some embodiments, the delivery device 30 may be advanced percutaneously through the vasculature to a position adjacent to the native heart valve 200 (e.g., the aortic valve) and/or the valve leaflets 210. For example, the delivery device 30 may be advanced through the vasculature and across the aortic arch to a position adjacent to the aortic valve. Alternative approaches to treat a defective aortic valve and/or other heart valve(s) are also contemplated with the delivery device 30. Orientation of the replacement heart valve implant 10, the delivery device 30, and/or elements thereof may be gleaned from FIGS. 1-3.

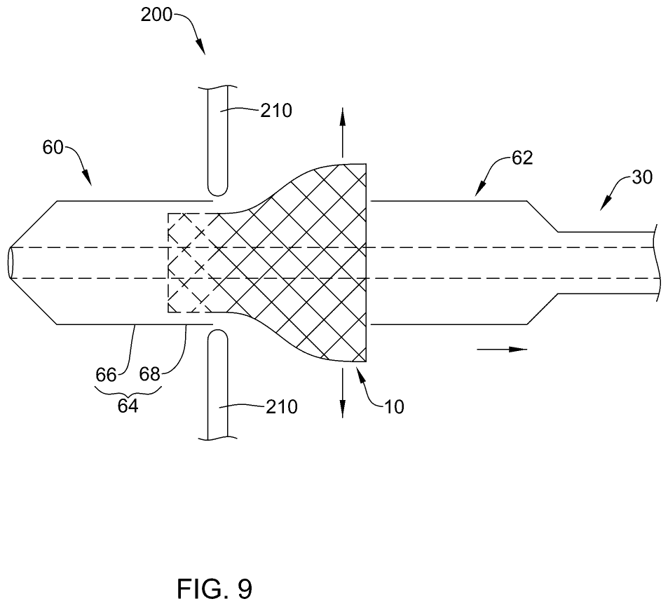

In some embodiments, the method of delivering the replacement heart valve implant 10 to the native heart valve 200 of the patient may comprise axially translating the proximal sheath 62 in the proximal direction relative to the replacement heart valve implant 10 and/or the intermediate tubular member 56 to release the proximal portion of the replacement heart valve implant 10 at and/or within the native heart valve 200, as seen in FIG. 9 (see also FIG. 3). In some embodiments, after positioning the replacement heart valve implant 10 and/or the expandable framework 12 at the treatment site and/or within the native heart valve 200, the first rotatable knob 43 may be rotated relative to the handle assembly 40 to axially translate the outer tubular member 52 and/or the proximal sheath 62 (e.g., the first portion of the elongate shaft assembly 50) proximally relative to the distal sheath 64 and/or the intermediate tubular member 56, thereby deploying the proximal portion of the replacement heart valve implant 10 from the implant holding portion 60 of the delivery device 30. Rotation of the first rotatable knob 43 in the second rotational direction may be configured to deploy the proximal portion of the replacement heart valve implant 10 from the implant holding portion 60 of the delivery device 30. In some embodiments, rotation of the first rotatable knob 43 in the second rotational direction to deploy the proximal portion of the replacement heart valve implant 10 from the implant holding portion 60 of the delivery device 30 may involve a series of slow, relatively short turns adding up to about 540 degrees of rotation, about 720 degrees of rotation, about 900 degrees of rotation, etc., using a pinch grip. Other configurations are also contemplated.

In some embodiments, after deploying the proximal portion of the replacement heart valve implant 10 from the implant holding portion 60 of the delivery device 30, positioning of the replacement heart valve implant 10 and/or the expandable framework 12 relative to the treatment site (e.g., the native heart valve, the aortic valve, etc.) may be verified using appropriate visualization means. In some embodiments, the replacement heart valve implant 10 may be recaptured and/or repositioned at and/or within the treatment site (e.g., the native heart valve, the aortic valve, etc.) as necessary.

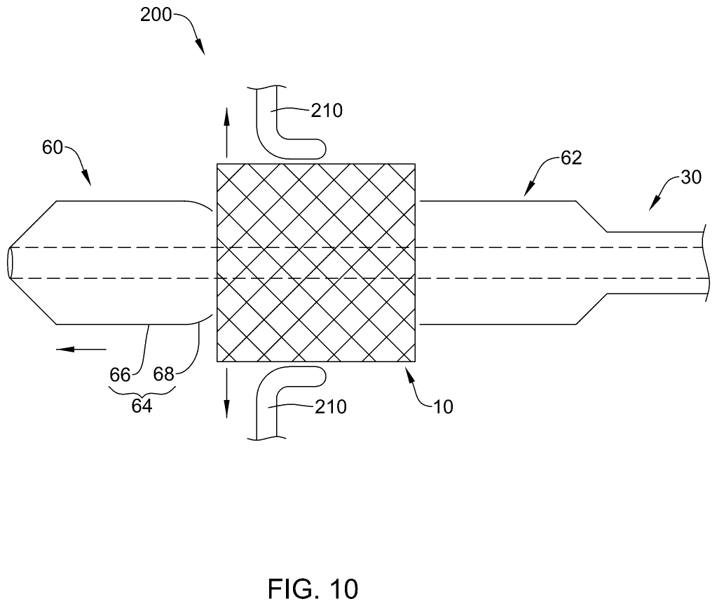

In some embodiments, the method of delivering the replacement heart valve implant 10 to the native heart valve 200 of the patient may comprise axially translating the distal sheath 64 in the distal direction relative to the replacement heart valve implant 10 and/or the intermediate tubular member 56 to release the distal portion of the replacement heart valve implant 10 at and/or within the native heart valve 200, as seen in FIG. 10 (see also FIG. 3). In some embodiments, after verifying the positioning of the replacement heart valve implant 10 and/or the expandable framework 12 relative to the native heart valve 200, and/or concluding that moving forward with the implantation procedure is desired, the second rotatable knob 44 may be rotated in the second rotational direction to translate the inner shaft 54 and/or the distal sheath 64 (e.g., the second portion of the elongate shaft assembly 50) distally relative to the proximal sheath 62 and/or the intermediate tubular member 56, thereby deploying the distal portion of the replacement heart valve implant 10 from the implant holding portion 60 of the delivery device 30. Rotation of the second rotatable knob 44 in the second rotational direction may be configured to deploy the distal portion of the replacement heart valve implant 10 from the implant holding portion 60 of the delivery device 30. In some embodiments, rotation of the second rotatable knob 44 in the second rotational direction to deploy the distal portion of the replacement heart valve implant 10 from the implant holding portion 60 of the delivery device 30 may involve a fast, relatively large degree of rotation using a power grip. In some embodiments, the relatively large degree of rotation may be between about 90 degrees and about 120 degrees, between about 95 degrees and about 115 degrees, between about 100 degrees and about 110 degrees, etc. In one non-limiting example, the relatively large degree of rotation may be about 103 degrees. Other configurations are also contemplated.

In some embodiments, the implant holding portion 60 may be configured to shift from the delivery configuration (e.g., FIGS. 2, 8) to the deployment configuration (e.g., FIGS. 3, 10) wherein the proximal sheath 62 and the distal sheath 64 are axially spaced apart by a distance greater than an overall length of the replacement heart valve implant 10 to release the replacement heart valve implant 10. In some embodiments, in the deployment configuration (e.g., FIGS. 3, 10) of the implant holding portion 60, the tapered portion 68 of the distal sheath 64 may be disposed distal of the replacement heart valve implant 10 and/or the proximal head 72 of the stent holder 70. In some embodiments, the tapered portion 68 of the distal sheath 64 may extend proximal of the body portion 76 of the stent holder 70 prior to releasing the replacement heart valve implant 10. In some embodiments, in the deployment configuration (e.g., FIGS. 3, 10) of the implant holding portion 60, the tapered portion 68 of the distal sheath 64 may be disposed in direct contact with the body portion 76 of the stent holder 70, as seen in FIGS. 3-4. In some embodiments, axially translating the distal sheath 64 in the distal direction relative to the replacement heart valve implant 10 and/or the intermediate tubular member 56 positions a proximalmost end of the tapered portion 68 of the distal sheath 64 in direct contact with the body portion 76 of the stent holder 70.

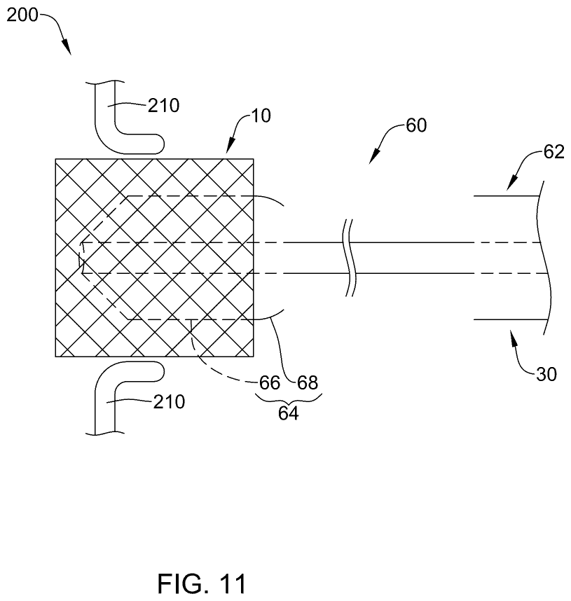

In some embodiments, the method of delivering the replacement heart valve implant 10 to the native heart valve 200 of the patient may comprise retracting the delivery device 30 relative to the replacement heart valve implant 10 until a proximal end of the distal sheath 64 and/or the proximal end of the tubular portion 66 of the distal sheath 64 is disposed proximal of the plurality of valve leaflets 20 of the replacement heart valve implant 10, as seen in FIG. 11. In some embodiments, the tapered portion 68 may be configured to deflect and/or direct the plurality of valve leaflets 20 of the replacement heart valve implant 10 around and/or toward and outside surface of the distal sheath 64 such that the distal sheath 64 cannot get caught (e.g., snag) on the plurality of valve leaflets 20 of the replacement heart valve implant 10.

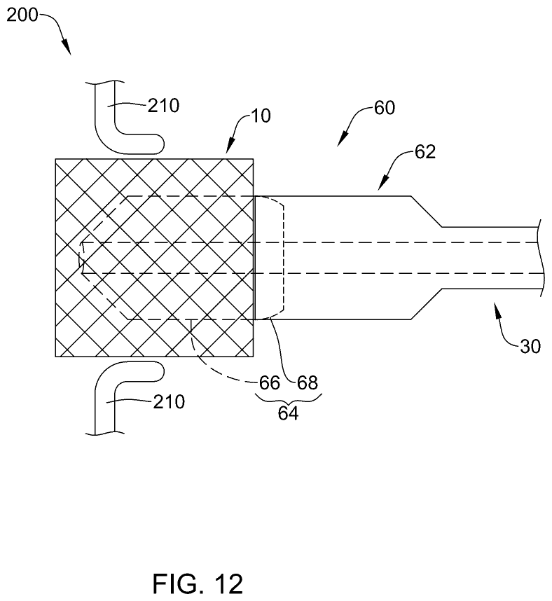

In some embodiments, the method of delivering the replacement heart valve implant 10 to the native heart valve 200 of the patient may comprise axially translating the proximal sheath 62 in the distal direction relative to the distal sheath 64 and/or the intermediate tubular member 56 until at least a portion of the proximal sheath 62 axially overlaps at least a portion of the distal sheath 64, as seen in FIG. 12. In some embodiments, the method of delivering the replacement heart valve implant 10 to the native heart valve 200 of the patient may comprise axially translating the proximal sheath 62 in the distal direction relative to the distal sheath 64 and/or the intermediate tubular member 56 (e.g., shifting the implant holding portion 60 to the withdrawal configuration). In some embodiments, axially translating the proximal sheath 62 in the distal direction relative to the distal sheath 64 and/or the intermediate tubular member 56 (e.g., shifting the implant holding portion 60 to the withdrawal configuration) positions a distalmost end of the proximal sheath 62 against an outwardly facing surface of the tapered portion 68 of the distal sheath 64. In some embodiments, axially translating the proximal sheath 62 in the distal direction relative to the distal sheath 64 and/or the intermediate tubular member 56 (e.g., shifting the implant holding portion 60 to the withdrawal configuration) positions a distalmost end of the proximal sheath 62 distal of a proximalmost end of the tapered portion 68 of the distal sheath 64. In some embodiments, axially translating the proximal sheath 62 in the distal direction relative to the distal sheath 64 and/or the intermediate tubular member 56 (e.g., shifting the implant holding portion 60 to the withdrawal configuration) axially overlaps a distalmost end of the proximal sheath 62 with the proximalmost end of the tapered portion 68 of the distal sheath 64.

In some embodiments, after retracting the delivery device 30 relative to the replacement heart valve implant 10 until the proximal end of the distal sheath 64 and/or the proximal end of the tubular portion 66 of the distal sheath 64 is disposed proximal of the plurality of valve leaflets 20 of the replacement heart valve implant 10, the first rotatable knob 43 may be rotated relative to the handle assembly 40 to axially translate the outer tubular member 52 and/or the proximal sheath 62 (e.g., the first portion of the elongate shaft assembly 50) distally relative to the distal sheath 64 and/or the intermediate tubular member 56, thereby shifting the implant holding portion 60 of the delivery device 30 toward and/or to the withdrawal configuration. Rotation of the first rotatable knob 43 in the first rotational direction may be configured to shift the implant holding portion 60 of the delivery device 30 toward and/or to the withdrawal configuration.

Thereafter, the method of delivering the replacement heart valve implant 10 to the native heart valve 200 of the patient may comprise removing the delivery device 30 from the patient. In some embodiments, removing the delivery device 30 from the patient may comprise positioning and/or retracting the delivery device 30 within a guide sheath, a positioning device, or other introducer, and subsequently removing both devices from the patient. Other configurations are also contemplated.

The materials that can be used for the various components of the implant delivery device and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the delivery device, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-NR and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof, or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A delivery device for delivering a replacement heart valve implant to a native heart valve, comprising:
a handle assembly; and
an elongate shaft assembly extending distally from the handle assembly, the elongate shaft assembly comprising:
an inner shaft secured to the handle assembly;
an outer tubular member disposed around the inner shaft and secured to the handle assembly; and
an implant holding portion comprising a proximal sheath and a distal sheath;
wherein in a delivery configuration of the implant holding portion, the implant holding portion is configured to constrain the replacement heart valve implant within the proximal sheath and the distal sheath;
wherein in a withdrawal configuration of the implant holding portion, at least a portion of the proximal sheath axially overlaps at least a portion of the distal sheath.

2. The delivery device of claim 1, wherein the proximal sheath is axially offset from the distal sheath in the delivery configuration.

3. The delivery device of claim 1, wherein the proximal sheath is axially non-overlapping with the distal sheath in the delivery configuration.

4. The delivery device of claim 1, wherein the distal sheath comprises a tubular portion having a constant inner diameter and a tapered portion disposed at a proximal end of the tubular portion, wherein an inner surface of the tapered portion tapers radially inward in a proximal direction.

5. The delivery device of claim 4, wherein an outer surface of the tapered portion tapers radially inward in the proximal direction such that at a proximalmost end of the tapered portion an outer diameter of the tapered portion is less than an inner diameter of the proximal sheath at a distalmost end of the proximal sheath.

6. The delivery device of claim 4, wherein the tapered portion comprises a plurality of longitudinally oriented spines formed from a different material than the tapered portion.

7. The delivery device of claim 1, wherein the implant holding portion is configured to shift from the delivery configuration to a deployment configuration wherein the proximal sheath and the distal sheath are axially spaced apart by a distance greater than an overall length of the replacement heart valve implant to release the replacement heart valve implant.

8. A delivery device for delivering a replacement heart valve implant to a native heart valve, comprising:
a handle assembly; and
an elongate shaft assembly extending distally from the handle assembly, the elongate shaft assembly comprising:
an inner shaft secured to the handle assembly;
an outer tubular member disposed around the inner shaft and secured to the handle assembly; and
an implant holding portion comprising a stent holder, a proximal sheath, and a distal sheath;
wherein the stent holder comprises a proximal head having a first outermost radial extent, a distal tail having a second outermost radial extent, and a body portion extending from the proximal head to the distal tail, the body portion having a constant outer diameter less than the first outermost radial extent and the second outermost radial extent;
wherein in a delivery configuration of the implant holding portion, the distal sheath is configured to constrain at least a portion of the replacement heart valve implant in engagement with the proximal head of the stent holder.

9. The delivery device of claim 8, wherein the distal sheath comprises a tubular portion having a constant inner diameter and a tapered portion disposed at a proximal end of the tubular portion, wherein the tapered portion is self-biased radially inward.

10. The delivery device of claim 9, wherein in the delivery configuration of the implant holding portion, the tapered portion is biased radially outward by the replacement heart valve implant.

11. The delivery device of claim 9, wherein in a deployment configuration of the implant holding portion, the tapered portion is disposed distal of the replacement heart valve implant and is in contact with the body portion of the stent holder.

12. The delivery device of claim 9, wherein in a withdrawal configuration of the implant holding portion, the proximal sheath is in contact with a radially outward facing surface of the tapered portion.

13. The delivery device of claim 9, wherein the tapered portion is formed from an elastomeric material.

14. The delivery device of claim 13, wherein the tubular portion comprises a reinforcing element configured to prevent radial expansion of the tubular portion.

15. The delivery device of claim 8, wherein in the delivery configuration of the implant holding portion, the proximal head of the stent holder is disposed within the distal sheath, and in a withdrawal configuration of the implant holding portion, the proximal head of the stent holder is disposed within the proximal sheath.

16. A method of delivering a replacement heart valve implant to a native heart valve of a patient, comprising:
advancing an implant holding portion of a delivery device, wherein the implant holding portion comprises a proximal sheath and a distal sheath constraining the replacement heart valve implant therein, to the native heart valve;
translating the proximal sheath in a proximal direction relative to the replacement heart valve implant to release a proximal portion of the replacement heart valve implant;
translating the distal sheath in a distal direction relative to the replacement heart valve implant to release a distal portion of the replacement heart valve implant;
translating the proximal sheath in the distal direction until at least a portion of the proximal sheath axially overlaps at least a portion of the distal sheath; and
removing the delivery device from the patient.

17. The method of claim 16, further comprising:
prior to translating the proximal sheath in the distal direction, retracting the delivery device relative to the replacement heart valve implant until a proximal end of the distal sheath is disposed proximal of valve leaflets of the replacement heart valve implant.

18. The method of claim 16, wherein the implant holding portion comprises a stent holder comprising a proximal head having a first outermost radial extent, a distal tail having a second outermost radial extent, and a body portion extending from the proximal head to the distal tail, the body portion having a constant outer diameter less than the first outermost radial extent and the second outermost radial extent.

19. The method of claim 18, wherein the distal sheath comprises a tubular portion and a tapered portion disposed at a proximal end of the distal sheath; and
wherein the tapered portion extends proximal of the body portion of the stent holder prior to releasing the replacement heart valve implant.

20. The method of claim 19, wherein translating the distal sheath in the distal direction positions a proximalmost end of the tapered portion in contact with the body portion of the stent holder.
